(19) United States
(12) Patent Application Publication
(10) Pub. No.: US 2008/0138406 A1
(43) Pub. Date: Jun. 12, 2008

(54) COMPOSITION FOR TREATMENT OF PAIN IN BONES AND JOINTS

(76) Inventor: Jarret Morrow, Boca Raton, FL (US)

Correspondence Address: ROBERT M. SCHWARTZ, P.A., P.O. BOX 221470, HOLLYWOOD, FL, 33022, US

(21) Appl. No.: 11/950,013

(22) Filed: Dec. 4, 2007

Related U.S. Application Data
(60) Provisional application No. 60/868,948, filed on Dec. 7, 2006.

(51) Int. Cl. A61K 36/00; A61K 31/35; A61K 31/195; A61K 9/48; A61K 9/20; A61K 9/28; A61K 9/52; A61K 9/24; A61P 43/00

(52) U.S. Cl. 424/457; 424/725; 514/459; 514/561; 424/451; 424/464; 424/463; 424/474; 424/472

(57) ABSTRACT

The present invention is a composition delivering effective amounts of Glucosamine, Devils Claw, and SAM in a single dosage unit.

This application claims the benefit of U.S. Provisional Patent Application Ser. No. 60/868,948 filed Dec. 7, 2006, the disclosure of which is incorporated herein by reference in its entirety.

### BRIEF SUMMARY OF THE INVENTION

The present invention relates to a novel combination of Glucosamine, Devils' Claw, and S-adenosyl methionine (SAM sometimes calles SAM-e). The dosage form provides greater efficacy than previous combinations of products.

Glucosamine, the name commonly used for 2-amino-2-deoxyglucose, 2-amino-2-deoxy-beta-D-glucopyranose (C₆H₁₃NO₅) is an amino sugar that is an important precursor in the biochemical synthesis of glycosylated proteins and lipids. It has the following structure:

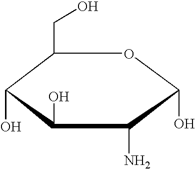

Oral glucosamine is commonly used for the treatment of osteoarthritis. Since glucosamine is a precursor for glycosaminoglycans, and glycosaminoglycans are a major component of joint cartilage, supplemental glucosamine is used to help to rebuild cartilage and treat arthritis. Typical oral dosage is 1500 mg/day.

Devil's Claw is from the plant Harpagophytum procumbens, also called grapple plant, wood spider, a plant of the sesame family, native to South Africa. It got its name from the peculiar appearance of its hooked fruit. The plant's large tuberous roots are used medicinally to reduce pain and fever, and to stimulate digestion.

The two active ingredients in Devil's Claw are Harpagoside and Beta Sitosterol. The British Herbal Pharmacopoeia recognises Devil's Claw as having analgesic, sedative and diuretic properties. It has been generally recognized that 50-100 mg/day of Harpagoside as a suggested dosage.

S-adenosyl methionine C₁₅H₂₄N₆O₅S (SAM) is a biological compound involved in methyl group transfers, and is present in all living cells. SAM is required for cellular growth and repair. It is also involved in the biosynthesis of several hormones and neurotransmitters that affect mood, such as epinephrine.

It has the following structural formula:

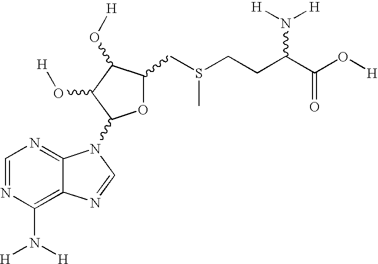

Because of structural instability, stable forms, as known in the art to be forms stable at room temerature over time, including molecular salt forms of SAM are required for its use as an oral drug. Although salt forms have been developed, SAM is still liable to degradation. Therapeutic doses, as practiced in the art, range from 800 mg/day to 1600 mg/day.

To combine these three substances, it would require a dosage form contining between 2350 mg-3200 mg of active ingredinets not including tableting excipients. This is an amount that would create a very large tablet size that would not be swallowable, or it would require formulation that would require ingesting multiple tablets to achieve the desired effect. It has been discovered that in certain combinations, the therapeutic amount of Glucosamine and SAM can be greatly reduced when certain ratios heretofore not recognized, of all three of these compounds combined into a single dosage form.

The present invention is a dosage form whereby Glucosamine, DC, and SAM (the ACTIVES)are combined into a single dosage form and the ratio between the ACTIVES create complimentary effects such that the threapeutic level of glucosamine and SAM are reduced.

This is advantageous because it becomes less expensive to provide the desired therapy and a single dosage form increases patient compliance with the therapy regimen.

In one embodiment the ratio of DC:Glucosamine:SAM is between 1:1-2:2-3. In a preferred embodiment, the ratio is 1:1.5:2.5. Still another preferred embodiment is 1:1.3:2.25. The increased amount of DC actuates the threrapeutic properties of both Glucosamine and SAM. When the ACTIVES are in combination, they provide for a single dosage form that allows threapy to ocurr at low dosage levels previously not recognized. There are many dosage forms known in the art, and detailed below. A preferred dosage form is a tablet. The amount of Glucosamine required is reduced 60 to 90% of the recognized therapeutic level. In one embodiment, the amount of Glucosamine in the dosage form is present at a level reduced by 75-90% of the recognized therapeutic level. In one embodiment, the amount of SAM is reduced 40 to 85% of the recognized therapeutic level. The reductions in these Glucosamine and SAM amounts allow the ACTIVES to be combined into a single dosage form while still providing the desired therapeutic effect.

In one embodiment the present invention comprises

An oral dosage form comprising or consisting of;
(a) Devils Claw;
(b) Glucosamine;
(c) SAM;
in a ratio of (a):(b):(C) of 1:1-2:2-3.

More preferably the dosage form has ratio of 1:1-1.5:2-2.5. In a preferred embodiment, Glucosamine is present in salt form that may be Glucosamine hydrochloride, Glucosamine sulfate, Glucosamine potassium sulfate, N-acetyl-Glucosamine or other acceptable salts.

In a preferred embodiment SAM is present in a salt form that may be any sulfate, phosphate, carbonate or other acceptable salts. A preferred salt is the disulfate p-toluenesulfonate.

The dosage form may be any dosage form acceptable for delivery of a therapeutic substance to a patient. The compositions can be provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a buccal or sublingual solid, a granule, a film, a sprinkle, a pellet, a bead, a pill, a powder, a triturate, a platelet, a strip or a sachet. Compositions can also be administered as a “dry syrup”, where the finished dosage form is placed directly on the tongue and swallowed or followed with a drink or beverage. These forms are well known in the art and are packaged appropriately. The compositions can be formulated for oral, nasal, buccal, or transmucosal, delivery, although oral delivery is presently preferred.

Most preferred is a dosage form that is a tablet or capsule.

In a preferred embodiment, the dosage form further comprises an enteric coating.

It is also preferred that the dosage form be provided as a single dosage unit.

It is an object of the present invention of the present invention to provide therapeutic levels of Glucosamine, DC, and SAM in a single dosage form.

### DETAILED DESCRIPTION OF THE PREFERRED EMBODIMENTS

The present invention provides for a dosage form delivering improved therapy of a combination of Glucosamine, DC, and SAM.

The compositions of the present invention can be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art.

The composition can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be affected by a polymeric matrix composition, a coated matrix composition, a multiparticulate composition, a coated multiparticulate composition, an ion-exchange resin-based composition, an osmosis-based composition, or a biodegradable polymeric composition.

The term “enteric coating” as used herein relates to a mixture of pharmaceutically acceptable excipients that is applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Alternatively, an enteric coating may be applied in an aqueous/organic cosolvent system. Additional additives and their levels, and selection of a primary coating material or materials will depend on the following properties: 1. resistance to dissolution and disintegration in the stomach; 2. impermeability to gastric fluids and drug/carrier/enzyme while in the stomach; 3. ability to dissolve or disintegrate rapidly at the target intestine site; 4. physical and chemical stability during storage; 5. non-toxicity; 6. easy application as a coating (substrate friendly); and 7. economical practicality.

Cellulose Derivatives are a preferred enteric coat material. Examples of suitable cellulose derivatives are: ethyl cellulose; reaction mixtures of partial acetate esters of cellulose with phthalic anhydride.

A preferred coating is aqueous Ethylcellulose Dispersion. The dispersion is a combination of film-forming polymer; plasticizer and stabilizers. Designed for sustained release and taste masking applications, the dispersion provides the flexibility to adjust drug release rates with reproducible profiles that are relatively insensitive to pH.

The principal means of drug release is by diffusion through the dispersion membrane and is directly controlled by film thickness. Increasing or decreasing the quantity of dispersion applied can easily modify the rate of release.

Two well-known dispersions are Surelease (Colorcon, West Point, PA) and Aquacoat ECD (FMC).

The performance of a coating can vary based on the degree and type of substitution. Cellulose acetate phthalate (CAP) dissolves in pH>6. Aquateric (FMC) is an aqueous based system and is a spray dried CAP psuedolatex. Other components in Aquateric can include pluronics, Tweens, and acetylated monoglycerides; cellulose acetate trimellitate (Eastman); methylcellulose (Pharmacoat, Methocel); hydroxypropyl methyl cellulose phthalate (HPMCP). The performance can vary based on the degree and type of substitution. HP-50, HP-55, HP-55S, HP-55F grades are suitable; hydroxypropyl methyl cellulose succinate (HPMCS; AQOAT (Shin Etsu)).

The coating can, and usually does, contain a plasticizer and possibly other coating excipients such as colorants, talc, and/or magnesium stearate, which are well known in the art. Suitable plasticizers include: triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the lower intestinal tract is reached.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

A coating process frequently involves spraying a coating solution onto a substrate. The coating solution can be a molten solution of the encapsulation coat composition free of a dispersing, medium. The coating solution can also be prepared by solubilizing or suspending the composition of the encapsulation coat in an aqueous medium, an organic solvent, a supercritical fluid, or a mixture thereof. At the end of the coating process, the residual dispersing medium can be further removed to a desirable level utilizing appropriate drying processes, such as vacuum evaporation, heating, freeze drying, etc.

Solvent-based coating is when the components of the invention are solubilized and/or dispersed in a solvent. The solvent can be aqueous. When the solvent is aqueous-based, the components can be emulsified with an appropriate emulsifier, organic solvent, or a supercritical fluid. Solvents with a lower melting point than water and higher evaporation numbers are preferred. Solvent mixtures with other organic solvents or water are often employed to get appropriate viscosity and component solubilization. Typical solvents include ethanol, methanol, isopropanol, acetone, dichloromethane, trichloromethane and ethyl acetate. Appropriate polymers can also be added as needed. Cellulosic derivatives and polymethacrylates are particularly suitable additives for organic solvent coating. Dissolution and solubilization of the components is facilitated by rigorous stirring or heating. Plasticizers may be also be added to stimulate dissolution. Colorants and antisticking agents can be employed as needed.

The following are presented by way of example and are not intended to limit the scope of the invention.

One general formulation is as follows:

| | |
|---|---|
| Glucosamine | 5-25% |
| DC | 3-15% |
| SAM | 10-40% |
| Filler | 20-75% |
| Binder | 1-20% |
| Disintegrant | up to 15% |
| Lubricant | up to 10% |
| Glident | up to 10% |

### EXAMPLE 1

In one embodiment a first blend comprising 100 g DC, 200 g Glucosamine, and 300 g of SAM are passed through a 25 mesh screen and blended until uniformly mixed. A second blend is prepared comprising 400 g microcrystalline cellulose (a common form sold as AVICELO by FMC, Philadelphia, Pa.),

54 g stearic acid, and 8 g croscarmellose sodium are each passed through a 25 mesh screen. The first and second blends are combined in a v-blender and mixed 45 minutes or long enough to ensure content uniformity as is commonly known and practiced in the art. The blender is stopped and 15 mg of silicon dioxide and 15 mg of magnesium stearate are screened through a 25 mesh screen and added to the blender. The mixture is blended an additional five minutes. The tableting mixture is discharged from the blender. Capsule shaped tablets with a target weight of 1300 mg (±6%) are compressed with a target hardness of 10-15 kP.

Tablets prepared according to Example 1 may optionally be coated with a layer. Alternatively, the tablets may be coated with more than one layer. Any layer may be functional or non-functional and may include, but would not be limited to controlled release, delayed release, sustained release, color, taste masking, moisture barrier, or any other layer disposed on the surface as are commonly practices in the art. In a preferred embodiment, the tablets are coated with an enteric layer such that they do not dissolve in the gastric pH of approximately 1.2.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. An oral dosage form comprising;
(a) Devils Claw;
(b) Glucosamine;
(c) SAM;
in a ratio of (a):(b):(C) of 1:1-2:2-3.

2. The dosage form of claim 1 having a ratio of 1:1-1.5:2-2.5.

3. The dosage form of claim 1 wherein Glucosamine is present as a salt, isomer, or derivative.

4. The dosage form of claim 1 wherein Glucosamine is glucosamine hydrochloride, glucosamine sulfate, glucosamine potassium sulfate, or N-acetyl-glucosamine.

5. The dosage form of claim 1 wherein SAM is present in a salt, isomer, or derivative.

6. The dosage form of claim 1 wherein SAM is present as a sulfate, phosphate, or carbonate.

7. The dosage form of claim 1 wherein SAM is present as the disulfate p-toluenesulfonate salt.

8. The dosage form of claim 1 wherein Devils Claw is present as salt, isomer, or derivative

9. The dosage form of claim 1 wherein said dosage form is a tablet or capsule.

10. The dosage form of claim 1 wherein said dosage form further comprises an enteric coating.

11. The dosage form of claim 1 wherein said dosage form is provided as a single unit.

12. The dosage form of claim 1 comprising:

| | | |
|---|---|---|
| (a) | Devils Claw | 3-15%; |
| (b) | Glucosamine | 5-25%; |
| (c) | SAM | 10-40%; |

based on the total weight of the dosage form.

13. The dosage form of claim 1 comprising a coating disposed on said dosage form.

14. The dosage form of claim 13 wherein said coating is at least one layer.

15. The dosage form of claim 13 wherein said layer is functional or non-functional.

16. The dosage form of claim 1 comprising a functional layer selected from controlled release, sustained release, delayed release, taste masking, or moisture control.

17. The dosage form of claim 1 comprising an enteric layer.

18. A method of providing therapy to a patient comprising the steps of:
(a) preparing a single unit dosage form comprising

| | |
|---|---|
| (i) | Devil's Claw; |
| (ii) | Glucosamine; |
| (iii) | SAM; |

said Devils Claw Glucosamine, and SAM being present in a ratio of 1:1-2:2-3 and being present in any form including salts, isomers, and derivatives;
(b) administering said single unit dosage form to a patient.
